(19) United States
(12) Patent Application Publication
(10) Pub. No.: US 2022/0395609 A1
(43) Pub. Date: Dec. 15, 2022

(54) ELECTROSPUN STRUCTURES FOR M1/M2 MACROPHAGE MODULATION AND METHODS OF MAKING AND USING THE SAME

(71) Applicant: Nanofiber Solutions, LLC, Dublin, OH (US)

(72) Inventors: Jed K. Johnson, London, OH (US); Ronald Lloyd Bracken, Monroe, GA (US)

(73) Assignee: Nanofiber Solutions, LLC, Dublin, OH (US)

(21) Appl. No.: 17/840,768

(22) Filed: Jun. 15, 2022

Related U.S. Application Data
(60) Provisional application No. 63/210,790, filed on Jun. 15, 2021.

(51) Int. Cl. A61L 27/18; A61L 27/50; A61L 27/26

(52) U.S. Cl. CPC A61L 27/18; A61L 27/50; A61L 27/26; A61L 2430/34; A61L 2430/22

(57) ABSTRACT

Disclosed herein are methods of controlling a composition of M1/M2 macrophages in a wound of a subject. The method can include applying an electrospun structure to the wound, wherein the electrospun structure comprises a polymer, wherein the polymer comprises an alpha-hydroxy acid, and keeping the electrospun structure on the wound for a time period. The presence of the electrospun structure on the wound causes an increase in presence of M2 macrophages relative to M1 macrophages, which can, in turn, promote healing of the wound.

### PRIORITY

The present application claims priority to U.S. Provisional Patent Application No. 63/210,790, titled ELECTROSPUN STRUCTURES FOR M1/M2 MACROPHAGE MODULATION AND METHODS OF MAKING AND USING THE SAME, filed Jun. 15, 2021, which is hereby incorporated by reference herein in its entirety.

### BACKGROUND

Beyond their impact in wound repair and remodeling, macrophages play an important role in regenerative medicine. Macrophages have been found to orchestrate critical processes of repair including granulation tissue formation, extracellular matrix remodeling, and angiogenesis. However, macrophage dysregulation is implicated in the pathologies of conditions including laryngotracheal stenosis, acute lung injury, fibrosis, excessive scarring, chronic wounds, and atherosclerosis. As a result, there is growing interest in macrophage-based therapies to modulate both macrophage infiltration and their phenotype. In one potential application, the trachea is populated with interstitial macrophages, which have recently been identified as distinct from their alveolar counterpart. These interstitial macrophages infiltrate the epithelial submucosa in response to injury; specifically, alternatively-activated M2 macrophages are upregulated and play an important role in epithelial repair.

Macrophage phenotypes exist on a spectrum, with M1 and M2 representing the two predominant subtypes. Macrophage polarization has implications in disease prognosis and can be influenced by the local microenvironment. Macrophage phenotype within a given microenvironment is often articulated in the form of a ratio between M1 and M2 subtypes. The M1/M2 ratio serves as an indicator for the influence of macrophages within a microenvironment on inflammation, tissue regeneration and repair. Recent studies have explored directed therapies to modulate the M1/M2 ratio to affect outcomes in tissue-engineered constructs, cellular therapies, and wound repair. Further, links between macrophages and angiogenesis have been demonstrated in the literature. See Corliss, Bruce A et al. “Macrophages: An Inflammatory Link Between Angiogenesis and Lymphangiogenesis,” Microcirculation (New York, N.Y.: 1994) vol. 23, issue 2 (2016): 95-121. doi:10.1111/micc.12259, which is hereby incorporated by reference herein in its entirety. Research has also indicated that increasing the presence of macrophages can improve the rate of wound healing, without negatively affecting the tissue repair quality. See Hu, Michael S et al. “Delivery of monocyte lineage cells in a biomimetic scaffold enhances tissue repair,” JCI insight vol. 2, 19 e96260. 5 Oct. 2017, doi:10.1172/jci.insight.96260, which is hereby incorporated by reference herein in its entirety. Therefore, devices that are configured to modulate the composition of M1/M2 macrophages at a wound site in order to affect angiogenesis, increase the rate of wound healing, and otherwise improve wound healing characteristics would be highly beneficial.

### SUMMARY

The present disclosure is directed to methods for controlling the presence or composition of M1 and/or M2 macrophages in a wound using electrospun structures, particularly to promote healing of the wound.

In some embodiments, there is provided a method of controlling a composition of M1/M2 macrophages in a wound of a subject, the method comprising: applying an electrospun structure to the wound, wherein the electrospun structure comprises a polymer, wherein the polymer comprises an alpha-hydroxy acid; and keeping the electrospun structure on the wound for a time period, wherein the presence of the electrospun structure on the wound causes an increase in presence of M2 macrophages relative to M1 macrophages.

In some embodiments, the electrospun structure comprises at least one polymer co-electrospun with the polymer.

In some embodiments, the at least one polymer comprises at least one of polyethylene terephthalate, polyurethane, polylactide co-caprolactone, polycaprolactone, or polylactic acid.

In some embodiments, the polymer comprises a resorbable polymer.

In some embodiments, the time period is two weeks.

In some embodiments, the electrospun structure comprises a graft.

In some embodiments, the electrospun structure comprises a patch.

In some embodiments, the polymer comprises polyglycolic acid.

In some embodiments, the increase in presence of the M2 macrophages relative to the M1 macrophages causes the wound to heal at an increased rate, increased angiogenesis at the wound, less scarring at the wound, or a combination thereof.

In some embodiments, the method further comprises combining the electrospun structure with a non-electrospun structure.

In some embodiments, the non-electrospun structure is selected from the group consisting of an allograft, a xenograft, a hernia mesh, or a suture.

In some embodiments, the alpha-hydroxy acid is selected from the group consisting of glycolic acid, lactic acid, and a combination thereof.

### FIGURES

FIG. 1 shows the repair of an anterior tracheal defect in a mouse using an electrospun patch, in accordance with at least one embodiment of the present disclosure.

FIG. 2 shows an immunohistochemistry analysis against macrophages (CD68+) M1 macrophages, and M2 macrophages to indicate macrophage infiltrates in the submucosa over PET/PU and PET/PU:PGA electrospun patches performed on serial axial sections, in accordance with at least one embodiment of the present disclosure.

FIG. 3 shows a series of graphs depicting the presence of macrophages, M1 macrophages, M2 macrophages, and the M1/M2 ratio at one week, two weeks, and six weeks post-implantation of the PET/PU and PET/PU:PGA electrospun patches, in accordance with at least one embodiment of the present disclosure.

### DETAILED DESCRIPTION

This disclosure is not limited to the particular systems, devices and methods described, as these may vary. The terminology used in the description is for the purpose of describing the particular versions or embodiments only and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “pharmaceutical” is a reference to one or more pharmaceuticals and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, the term “M1 macrophage” refers to the classically activated phenotype of macrophages, including macrophages that are typically activated interferon gamma (IFN-γ) or lipopolysaccharide (LPS) and produce pro-inflammatory cytokines, phagocytize microbes, and initiate an immune response. As used herein, the term “M2 macrophage” refers to the alternatively activated phenotype of macrophages, including macrophages that are typically activated by other (i.e., not IFN-γ) cytokines (e.g., various interleukins (ILs), including IL-4, IL-10, or IL-13) and produce either polyamines to induce proliferation or proline to induce collagen production.

As used herein, the term “improve” is used to convey that the methods of healing tissues as described in embodiments herein change the appearance, form, characteristics and/or the physical or biochemical attributes of the tissues to which they are being provided, applied or administered. Suitably, such changes are beneficial changes, for example in relation to the appearance, form, characteristics and/or the physical or biochemical attributes of the tissues to which the scaffold is applied.

The terms “heal,” “treat,” “treated,” or “treating,” as used herein, refers to both therapeutic treatment and prophylactic or preventative measures, wherein the object is to inhibit, prevent or slow down (lessen) an undesired physiological condition, disorder or disease, or to accelerate, improve, inhibit, or otherwise obtain beneficial or desired clinical results. For the purposes of this invention, beneficial or desired clinical results include, but are not limited to, full or partial healing of a wound or damaged tissue; the decrease in size or planimetric area of a wound; decreased inflammation of a wound or damaged tissue; prevention of further wound development or tissue damage; improvement or alleviation of symptoms, including pain or swelling; or decreased scarring resulting from the healing of the wound or damaged tissue. Accelerating healing or treating of a wound refers to increasing the rate of onset of a beneficial effect as described above, compared to the rate of onset without use of the scaffold.

As used herein, “applying to a wound” means any method of contacting the scaffold with all or a portion of a wound. Application may include placing a scaffold on the surface of a wound, and/or implanting a scaffold within a body to make contact with a wound. Suitably, direct contact is made between the scaffold and wound, although it is also envisaged that contact may be indirect, for example where an intervening material, substance or composition is present.

As used herein, the term “subject” includes, but is not limited to, humans, non-human vertebrates, and animals such as wild, domestic, and farm animals. In some embodiments, the term “subject” refers to mammals. In some embodiments, the term “subject” refers to humans. A “tissue” may include any cell or collection of cells within a subject.

As used herein, the terms “wound” and “damaged tissue” may be used interchangeably. A wound or damaged portion of tissue may be located anywhere within a subject's body, either internal or external. A wound or damaged portion of tissue may occur as the result of trauma, surgery, pressure, friction, or a non-traumatic occurrence.

As used herein, the term “chronic wound,” which may be used interchangeably herein with the term “non-healing wound,” describes a wound or damaged portion of tissue that heals more slowly than a typical wound of the same type. A chronic wound may, for example, fail to heal in the orderly stages in which a typical wound might heal. A chronic wound may also fail to heal within an expected period of time, for a variety of reasons. In other words, a chronic wound is one that may remain in a particular phase of healing for too long, such as the inflammatory phase. One example of a chronic wound is one that does not heal within three months of its development or creation. Chronic wounds may result from factors including pressure, trauma and/or lower extremity wounds, increased bacterial load, excessive proteases, degraded growth factors, matrix metalloproteinases (MMPs), degraded cell surface structures, senescent/aberrant cells and inappropriate treatment. Examples of chronic wounds include ulcers such as venous ulcers, diabetic ulcers, and, pressure ulcers, ischemia, and wounds resulting from radiation poisoning.

### Electrospinning

Electrospinning is a method which may be used to process a polymer solution into a structure, such as a fiber. In embodiments where the diameter of the resulting fiber is on the nanometer scale, the fiber may be referred to as a nanofiber. Fibers may be formed into a variety of shapes by using a range of receiving surfaces, such as mandrels, molds, or collectors. The resulting fiber molds or shapes may be used in many applications, including the repair or replacement of biological structures. In some embodiments, the resulting structure (e.g., a fiber or fiber scaffold) may be implanted into a biological organism or a portion thereof.

Electrospinning methods may involve spinning a structure (e.g., a fiber) from a polymer solution by applying a high DC voltage potential between a polymer injection system and a receiving surface. In some embodiments, one or more charges may be applied to one or more components of an electrospinning system. In some embodiments, a charge may be applied to the receiving surface, the polymer injection system, the polymer solution, or combinations or portions thereof. Without wishing to be bound by theory, as the polymer solution is ejected from the polymer injection system, it is thought to be destabilized due to its exposure to a charge. The destabilized solution may then be attracted to a charged receiving surface. As the destabilized solution moves from the polymer injection system to the receiving surface, its solvents may evaporate, and the polymer may stretch, leaving a long, thin fiber that is deposited onto the receiving surface. The polymer solution may form a Taylor cone as it is ejected from the polymer injection system and exposed to a charge. Further, polymers can be electrospun in a variety of different structures, including fibers, fibrous scaffolds, strips, patches, sheets, or shapes corresponding to anatomical structures. Still further, the structures can be electrospun using one or multiple polymers.

In some embodiments, multiple polymer types can be electrospun with each other to form structures in a process referred to as “co-electrospinning.” In co-electrospinning, two or more polymer solutions (containing the same or different polymer types) are ejected from different outlets and simultaneously electrospun with each other to form the resultant structure. Co-electrospinning creates two different fibers formed from the different polymer solutions that are intertwined with each other. The co-electrospun polymers can be spun from the same or different polymer solutions. The co-electrospun polymer types can have the same or different degradation rates. As one example, a first polymer type having a first degradation rate could be co-electrospun with a second polymer type having a second degradation rate, thereby creating an electrospun structure providing a time-released profile that releases one or more pharmaceuticals contained within the different polymer types based on the differing degradation rates of the polymer types.

In some embodiments, multiple polymer types can be electrospun with each other to form structures in processes referred to as “coaxial electrospinning” or “multiaxial electrospinning.” In coaxial or multiaxial electrospinning, two or more polymer solutions (containing the same or different polymer types) are rejected from the same outlet and electrospun with each other to form the resultant structure. Coaxial or multiaxial electrospinning creates a single fiber composed of the different polymer types that has a core-shell structure. The coaxially or multi-axially electrospun polymer types can have the same or different degradation rates. As one example, a first polymer type having a first degradation rate could be coaxially or multi-axially electrospun with a second polymer type having a second degradation rate, thereby creating an electrospun structure providing a time-released profile that releases one or more pharmaceuticals contained within the different polymer types based on the differing degradation rates of the polymer types.

In some embodiments, the co-electrospinning and coaxial or multiaxial electrospinning techniques described above could also be used in combination with each other. For example, coaxial polymers or fibers could be co-electrospun with each other. Accordingly, the various techniques described above can be used to electrospin structures having various timed release profiles for pharmaceuticals (which are described below) contained therein.

### Polymer Injection System

A polymer injection system may include any system configured to eject some amount of a polymer solution into an atmosphere to permit the flow of the polymer solution from the injection system to the receiving surface. In some embodiments, the polymer injection system may deliver a continuous or linear stream with a controlled volumetric flow rate of a polymer solution to be formed into a structure (e.g., a fiber). In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with one or more syringes containing one or more different polymer solutions. In some embodiments, the polymer injection system could include a rotating drum that dips into the polymer solution and ejects the solution as the drum rotates. In some embodiments, the polymer injection system could include a wire-based electrospinning system. In some embodiments, a charge may be applied to the polymer injection system or to a portion thereof. In some embodiments, a charge may be applied to a needle or needle-like component of the polymer injection system. In one particular embodiment, the polymer injection system could include a wire electrode-based polymer injection system, such as the NS 8S1600U electrospinning production line available from ELMARCO®.

In some embodiments, the polymer solution may be ejected from the polymer injection system at a flow rate per needle of less than or equal to about 5 mL/h. Some non-limiting examples of flow rates per needle may include about 0.1 mL/h, about 0.5 mL/h, about 1 mL/h, about 1.5 mL/h, about 2 mL/h, about 2.5 mL/h, about 3 mL/h, about 3.5 mL/h, about 4 mL/h, about 4.5 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any ranges between any two of these values, including endpoints.

As the polymer solution travels from the polymer injection system toward the receiving surface, the diameter of the resulting fibers may be in the range of about 0.1 μm to about 10 μm. Some non-limiting examples of electrospun fiber diameters may include about 0.1 μm, about 0.2 μm, about 0.5 μm, about 1 μm, about 2 μm, about 5 μm, about 10 μm, about 15 μm, about 20 μm, or ranges between any two of these values, including endpoints.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include synthetic or semi-synthetic polymers such as, without limitation, poly(ethylene oxide), polyvinyl pyrrolidone, Dextran, saccharide, cellulose, chitosan, gelatin, collagen, polyvinyl alcohol, Eudragit, polyethylene terephthalate (PET), polyester, polymethylmethacrylate, polyacrylonitrile, silicone, polyurethane (PU), polycarbonate, polyether ketone ketone, polyether ether ketone, polyether imide, polyamide, polystyrene, polyether sulfone, polysulfone, polyvinyl alcohol (PVA), polyvinyl acetate (PVAc), polycaprolactone (PCL), polylactic acid (PLA), polylactide co-caprolactone, polylactide co-glycolide, polyglycolic acid (PGA), polyglycerol sebacic, polydiol citrate, polyhydroxy butyrate, polyether amide, polydioxanone, copolymers thereof, and combinations or derivatives thereof. In some embodiments, the polymer solution may include a polymer that is a water-soluble polymer. Alternative polymer solutions used for electrospinning may include natural polymers such as fibronectin, collagen, gelatin, hyaluronic acid, chitosan, or combinations thereof. It may be understood that polymer solutions may also include a combination of synthetic polymers and naturally occurring polymers in any combination or compositional ratio. In some non-limiting examples, the polymer solution may comprise a weight percent ratio of, for example, poly(ethylene oxide) to polycaprolactone, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer may be present in an amount of about 1 wt % to about 30 wt % based on the weight of the polymer solution. In some non-limiting examples, the polymer may be present in the amount of, for example, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, or ranges between any two of these values, including endpoints.

In some embodiments, the polymer solution may comprise one or more solvents. In some embodiments, the solvent may comprise, for example, acetone, dimethylformamide, dimethylsulfoxide, N-methylpyrrolidone, N,N-dimethylformamide, acetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. Non-limiting examples of alcohols include ethanol, isopropanol, butanol, and the like. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

The type of polymer in the polymer solution may determine the characteristics of the electrospun structure. Some structures may be composed of polymers that are bio-stable and not absorbable or biodegradable when implanted. Such structures may remain generally chemically unchanged for the length of time in which they remain implanted. Alternatively, structures may be composed of polymers that may be absorbed or biodegraded over time. Such structures may act as an initial template or scaffold for the repair or replacement of organs and/or tissues. These organ or tissue templates or scaffolds may degrade in vivo once the tissues or organs have been replaced or repaired by natural structures and cells. Alternatively, such structures may degrade or disintegrate at a faster controlled rate, such as a rate appropriate for drug delivery rather than cell or tissue ingrowth. It may be further understood that a polymer solution and its resulting electrospun structure(s) may be composed of more than one type of polymer, and that each polymer therein may have a specific characteristic, such as bio-stability or biodegradability.

### Applying Charges to Electrospinning Components

In an electrospinning system, one or more charges may be applied to one or more components, or portions of components, such as, for example, a receiving surface, a polymer injection system, a polymer solution, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to the polymer solution, or portions thereof. In some embodiments, a negative charge may be applied to the polymer solution, or portions thereof. In some embodiments, the polymer solution, or portions thereof, may be grounded. In some embodiments, a positive charge may be applied to the receiving surface, or portions thereof. In some embodiments, a negative charge may be applied to the receiving surface, or portions thereof. In some embodiments, the receiving surface, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −100 kV to about 100 kV, including endpoints. In some non-limiting examples, the charge applied to any component of the electrospinning system, or portions thereof, may be about −100 kV, about −75 kV, about −50 kV, about −30 kV, about −25 kV, about −15 kV, about −10 kV, about −5 kV, about −3 kV, about −1 kV, about −0.01 kV, about 0.01 kV, about 1 kV, about 5 kV, about 10 kV, about 12 kV, about 15 kV, about 20 kV, about 25 kV, about 30 kV, about 50 kV, about 75 kV, about 100 kV, or any range between any two of these values, including endpoints. In some embodiments, any component of the electrospinning system, or portions thereof, may be grounded.

### Receiving Surface Movement During Electrospinning

During electrospinning, in some embodiments, the receiving surface may move with respect to the polymer injection system. In some embodiments, the polymer injection system may move with respect to the receiving surface. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the receiving surface may be in contact with or mounted upon a support structure that may be moved using one or more motors or motion control systems. The pattern of the electrospun structure deposited on the receiving surface may depend upon the one or more motions of the receiving surface with respect to the polymer injection system. In some embodiments, the receiving surface may be configured to rotate about its long axis. In one non-limiting example, a receiving surface having a rotation rate about its long axis that is faster than a translation rate along a linear axis may result in a nearly helical deposition of an electrospun fiber, forming windings about the receiving surface. In another example, a receiving surface having a translation rate along a linear axis that is faster than a rotation rate about a rotational axis may result in a roughly linear deposition of an electrospun fiber along a linear extent of the receiving surface. In some embodiments, the electrospinning system could include a roller electrospinning system.

### M1/M2 Macrophage Modulation

The present disclosure is directed to modulating or controlling the presence of M1 and/or M2 macrophages in or at a wound (e.g., a chronic wound or a surgically created wound), particularly the ratio of M1 to M2 macrophages, using electrospun structures. It should be understood that the devices and methods described herein may be applied to the treatment of wounds (e.g., chronic wounds or surgical wounds) and any medical procedure and that the examples described herein are non-limiting.

In some embodiments, the method can include applying an electrospun structure to a wound for a time period, wherein the electrospun structure is configured to modulate or control the composition of M1 and M2 macrophages at or in the wound. In one embodiment, the electrospun structure can be configured to increase a presence of M2 macrophages relative to M1 macrophages. In another embodiment, the electrospun structure can be configured to decrease the presence of M1 macrophages relative to M2 macrophages. Without wishing to be bound by theory, reducing the ratio of M1/M2 macrophages can be beneficial because M1 macrophages produce pro-inflammatory cytokines, which can result in increased inflammation and scarring in the wound and can reduce the ability of the wound to heal over time.

In one embodiment, the structure can be electrospun from a polymer or a copolymer comprising an alpha-hydroxy acid. The alpha-hydroxy acid could include, for example, glycolic acid or lactic acid. The polymer comprising the alpha-hydroxy acid could further include any derivative or copolymer thereof. For example, the polymer could include polyglycolic acid (PGA) or poly(lactic-co-glycolic acid) (PLGA). As another example, polymer could include poly(lactic acid) (PLA), poly-L-lactide (PLLA), or poly-D-lactide (PDLA). In one embodiment, the polymer can comprise a resorbable polymer. In one embodiment, the polymer can comprise a non-resorbable polymer. In one embodiment, the structure can be co-electrospun from two or more polymers, wherein at least one of the two or more polymers comprises an alpha-hydroxy acid. In this embodiment, the other polymer(s) (i.e., the polymer(s) not comprising the alpha-hydroxy acid) can comprise, without limitation, any of the polymers described above. In some embodiments, the structure can be electrospun from the one or more polymers using any of the electrospinning methods or techniques described above.

In some embodiments, the electrospun structure can have a thickness from about 50 μm to about 2 mm. The thickness of the electrospun structure may be, for example, about 50 μm, about 60 μm, about 70 μm, about 80 μm, about 90 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1 mm, about 2 mm, or any range between any two of these values, including endpoints.

In some embodiments, the electrospun structure may have a length from about 1 cm to about 20 cm. The length of the electrospun structure may be, for example, about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, or any range between any two of these values, including endpoints.

In some embodiments, the electrospun structure may have a width from about 1 cm to about 20 cm. The width of the electrospun structure may be, for example, about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, or any range between any two of these values, including endpoints.

In some embodiments, the electrospun structure can be electrospun into a variety of different shapes or configurations, such as, for example, a graft, a patch, a fragment, a cluster, a strand, a thread, a rope, a braid, a sheet, a coil, a tube, a cylinder, a textile, or a mold of an organ. In some embodiments, the structure may be formed into a mold of an organ such as, for example, a trachea, a trachea and at least a portion of at least one bronchus, a trachea and at least a portion of a larynx, a larynx, an esophagus, a large intestine, a small intestine, an upper bowel, a lower bowel, a vascular structure, an artery, a vein, a nerve conduit, a ligament, a tendon, and portions thereof. In some embodiments, the structure may be formed into the shape of a suture.

In some embodiments, the electrospun structure could include a sheet, strip, or patch. In some embodiments, the electrospun structures could have an average length of about 1 cm to about 6 cm, an average width of about 1 cm to about 6 cm, and an average thickness of about 1 mm to about 2 mm.

In one embodiment, the time period could be two weeks. The time period may be, for example, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, or any range between any two of these values, including endpoints.

In some embodiments where the electrospun structure comprises multiple polymers, the polymers can be co-electrospun, coaxially electrospun, and/or sequentially electrospun, as described above. Accordingly, the configuration of the electrospun structure can be controlled based on the manner or technique used to electrospun the polymers to form the structure.

In some embodiments where the electrospun structure comprises multiple polymers, each of the different polymers can be selected to have different degradation time periods or may comprise a non-resorbable polymer. For example, the electrospun structure can comprise a first polymer having a first degradation time period and a second polymer having a second degradation time period. It may be beneficial to electrospin the structure using polymers with different degradation times in order to control the manner and/or rate at which the electrospun structure degrades.

In some embodiments, the electrospun structure can be combined with non-electrospun materials or structures, such as an allograft, a xenograft, a hernia mesh, or a suture. In various embodiments, the non-electrospun materials or structures can be partially or wholly enclosed by the electrospun structure, attached to the electrospun structure via various attachment modalities (e.g., bioadhesives, heat welding, or sutures), or otherwise incorporated into the electrospun structure. For example, the electrospun structures described herein could be fabricated to at least partially enclose or encase an allograft. As another example, the electrospun structures described herein could be attached to a hernia mesh. Various techniques for incorporating non-electrospun materials or structures into electrospun structures are described in U.S. patent application Ser. No. 16/292,026, titled BONE GROWTH FACILITATION DEVICE AND METHODS OF USE, filed Mar. 4, 2019, which is hereby incorporated by reference herein in its entirety.

In some embodiments, after the time period has passed, the wound will have a decreased planimetric area. The decrease in the planimetric area of the wound will correspond to the treatment of the wound. The wound may have, for example, a decrease in planimetric area of about 1% to about 100%. The decrease in planimetric area may be, for example, about 1%, about 5%, about 10%, about 20%, about 30%, about 40%, about 50%, about 60%, about 70%, about 80%, about 90%, about 100%, or any range between any two of these values, including endpoints.

In some embodiments, the method can further include applying a second electrospun structure, as described herein, to the wound to further control or modulate the presence of M1 and/or M2 macrophages. The method may also comprise keeping the second electrospun structure on the wound for a second time period, which may correspond to the first time period described herein. After the second time period, the chronic wound may have a decreased planimetric area.

In some embodiments, the step of applying an electrospun structure to the wound can be performed repeatedly. In other words, the method can further include applying a third electrospun structure, a fourth electrospun structure, a fifth electrospun structure, a sixth electrospun structure, and so on. Further, the method can further include keeping the electrospun structure applied to the wound for a third time period, a fourth time period, a fifth time period, a sixth time period, and so on. In some embodiments, the method may also comprise trimming or shaping the electrospun structure to fit the wound or using multiple electrospun structures to cover a single wound.

As noted above, the electrospun structures can degrade over time when in contact with the subject's body (e.g., the wound). Without wishing to be bound by theory, a degradation byproduct of PGA or other polymers comprising an alpha-hydroxy acid (e.g., glycolic acid) may result in an increased expression of M2 macrophages relative to M1 macrophages at or in a wound. The increased expression of M2 macrophages can reduce the relative signaling effects resulting from the expression of M1 macrophages, such as an inflammatory response. Further, a reduced inflammatory response may promote healing of the wound and less scarring or fibrosis. Accordingly, applying electrospun structures to a wound that are configured to reduce the M1/M2 ratio can be beneficial in order to promote wound healing. The improvement in the healing of a wound due to the increased M1/M2 ratio can be embodied by, for example, a reduced planimetric area of the wound, an increase rate of healing of the wound, an increased rate of angiogenesis at the wound site, or a reduction in scarring resulting from the healing of the wound.

### EXAMPLES

The examples discussed below are provided solely in order to further illustrate benefits and potential applications of the techniques described above. They should not be understood or be used to further limit the various embodiments described above.

### Example 1: Tissue Engineered Tracheal Graft

In one embodiment, tracheal scaffolds made from electrospun PET/PU and co-electrospun PET/PU with PGA having a 300 μm wall thickness were manufactured as previously described. The scaffolds were then plasma treated, packaged, and sterilized by UV illumination at 35 J/cm². The performance of electrospun tracheal scaffolds was experimentally compared to (i) syngeneic trachea grafts (STGs) harvested from six to eight-week-old female C57BL/6J mice euthanized pharmacologically and mechanically with bilateral pneumothoraces and (ii) decellularized tracheal scaffolds collected in a manner similar to the STGs and then subjected to a graded sodium dodecyl sulfate (SDS) treatment before immersion in 1% TritonX-100 for 30 min at room temperature and 0.9% NaCl solution wash overnight at 4° C.

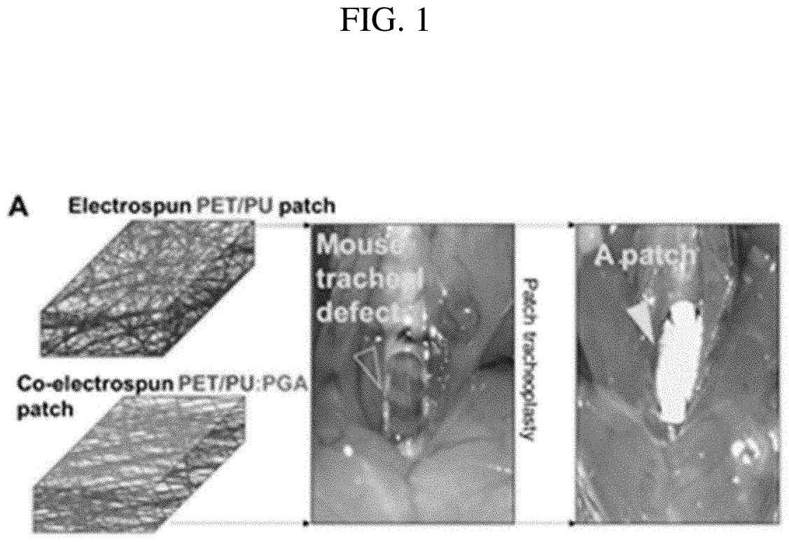

To assess macrophage infiltration during “normal” tracheal repair, animals (n=20) were randomly assigned to 1 week, 2 weeks, 1 month, 6 months and 1 year time points following tracheal replacement with STG (n=4/time point). This was designed to account for early drop out from an overall survival rate of >80%. Slides were inspected for quality and three grafts were randomly selected from each group. A mouse model of patch tracheoplasty was used to evaluate synthetic grafts. Animals were randomly assigned to time points of 1, 2 and >6 weeks (n=7/scaffold type/time point). Of this cohort, 36/42 animals survived to planned endpoint. Segmental tracheal replacement with DTS was performed on animals with the endpoints of 1 month (n=12) and 3 months (n=15). Overall survival to planned endpoint was 44%. Histologic sections were assessed for quality and four animals per time point were randomly selected for characterization. Procedures were performed in 6-8 week old, female C57BL/6J mice as previously described. All procedures were done under general anesthesia with aseptic technique. During orthotopic replacements with STG and DTS, a 3-4 mm long segment of the host trachea was excised and replaced with the graft of interest. For synthetic TETG recipients, a 1×2 mm portion of the anterior tracheal wall was excised and replaced by a size-matched patch of electrospun PET/PU or PET/PU:PGA, as shown in FIG. 1. In particular, FIG. 1 shows the repair of an anterior tracheal defect (empty arrow head) with a synthetic patch (filled arrow head) made of either electrospun PET/PU or co-electrospun PET/PU:PGA.

At endpoint (either experimental or humane), animals were administered an intraperitoneal overdose of ketamine/xylazine cocktail (200 mg/kg ketamine, 20 mg/kg xylazine, 10 mg/kg ketoprofen). Upon euthanasia, grafts were harvested and fixed in 10% neutral buffered formalin for at least 48 hours at room temperature before histological processing.

Host macrophages were depleted via intraperitoneal injection of clodronate (CCL) liposomes (5 mg/mL) (n=3) and PBS liposomes (n=3) in animals at 2-day intervals beginning 3 days pre-implantation through endpoint (post-operative days 4 and 14). Systemic macrophage depletion was confirmed using flow cytometry. In brief, bone marrow was extracted from mice femur and long bones. Extracted cells were treated with RBC lysis buffer (Thermo Fisher, Mass., USA). Cells were blocked with Fc-block and stained with Live/Dead-NIR (Thermo Fisher), CD45-BV510 (Biolegend, San Diego, Calif., USA), Ly6C-PE (Biolegend), and F4/80-BV421 (Biolegend), fixed with 4% paraformaldehyde, then sorted using BD LSRFortessa. Data analysis was performed using the FloJo software (FlowJo LLC, Ashland, Oreg.).

To assess the graft-host interface, segmental grafts (STG and DTS) were sectioned longitudinally and the patch grafts (synthetic TETG) were sectioned axially (4 μm) with 3 sections per slide. Immunofluorescence staining was performed to identify basal progenitor cells (K5+) and ciliated epithelial cells (ACT+).

Immunohistochemical staining to identify macrophages and macrophage phenotypes using adjacent sections of the same slide was performed. Primary antibodies were diluted in Dako Antibody Diluent (Agilent). Secondary antibody binding was achieved with incubating a 1:1500 dilution of goat anti-rabbit IgG biotinylated antibodies (Vector) for 30 minutes before binding of horseradish peroxidase conjugated with streptavidin (Vector). Sections were developed with 3,3-diaminobenzidine (DAB) and counterstained with Gill's hematoxylin.

Macrophage density (cells/mm²) over the graft or patch and the host tracheal section was calculated using ImageJ software. The M1/M2 ratio was measured by dividing the density of M1 macrophages (iNOS+) by the density of M2 macrophages (CD206+) in adjacent sections.

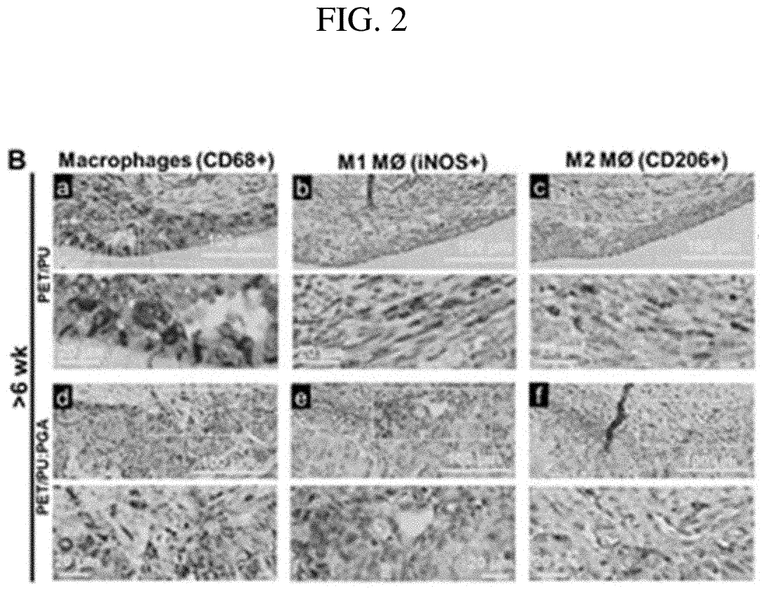

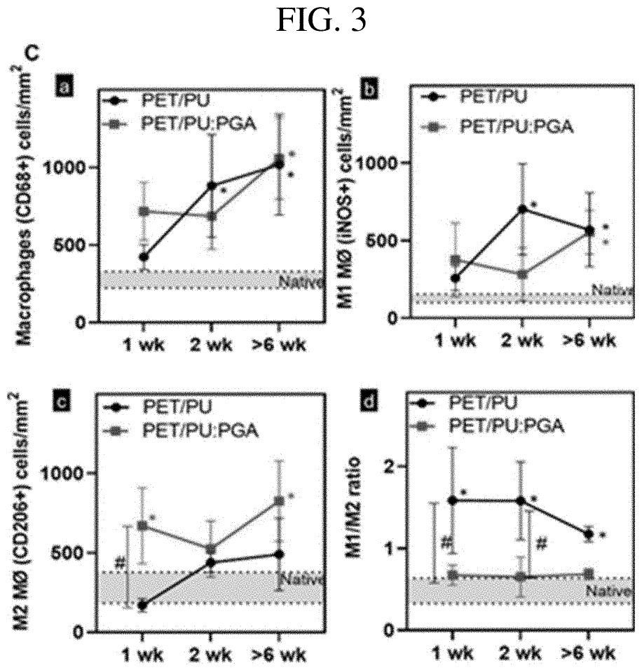

The resulting analysis of the samples found that implants of both the PET/PU and PET/PU:PGA graft types resulted in an elevated macrophage infiltrate in the epithelial submucosa compared to native, which did not differ between PET/PU and PET/PU:PGA grafts, as is shown in FIG. 2. However, macrophage dysregulation was observed in PET/PU grafts, as represented by an increase of the M1/M2 ratio (1.44±0.23). Graft modification with the addition of co-electrospun PGA led to a shift of M1/M2 ratio to 0.67±0.01, equivalent to the macrophage ratio of native trachea, as shown in FIG. 3. In particular, FIG. 3 includes a first graph (a) depicting the presence of macrophages (CD68+) per mm² at various time points, a second graph (b) depicting the presence of M1 macrophages (MØ) (iNOS+) per mm² at the time points, a third graph (c) depicting the presence of M2 MØ (CD206+) per mm² at the time points, and a fourth graph (d) depicting the corresponding M1/M2 ratio at the time points in the patch. Macrophage counts in native phenotypes are indicated by the grey bars. As can be seen in FIG. 3, particularly the fourth graph (d) thereof, the data suggests that that the addition of PGA shifted the composition of macrophages towards the M2 phenotype.

In sum, elevation of the M1/M2 ratio was observed with synthetic (i.e., electrospun) tracheal replacement. Further, macrophage composition in the grafts was able to be modulated without altering macrophage levels via scaffold biomaterial selection and host macrophage depletion.

While the present disclosure has been illustrated by the description of exemplary embodiments thereof, and while the embodiments have been described in certain detail, it is not the intention of the Applicant to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1. A method of controlling a composition of M1/M2 macrophages in a wound of a subject, the method comprising:
applying an electrospun structure to the wound, wherein the electrospun structure comprises a first polymer, wherein the first polymer comprises an alpha-hydroxy acid; and
keeping the electrospun structure on the wound for a time period, wherein the presence of the electrospun structure on the wound causes an increase in a presence of M2 macrophages relative to M1 macrophages.

2. The method of claim 1, wherein the electrospun structure comprises at least one second polymer co-electrospun with the first polymer.

3. The method of claim 2, where the at least one second polymer comprises at least one of polyethylene terephthalate, polyurethane, polylactide co-caprolactone, polycaprolactone, or polylactic acid.

4. The method of claim 1, wherein the first polymer comprises a resorbable polymer.

5. The method of claim 1, wherein the time period is two weeks.

6. The method of claim 1, wherein the electrospun structure comprises a graft. The method of claim 1, wherein the electrospun structure comprises a patch.

8. The method of claim 1, wherein the first polymer comprises polyglycolic acid.

9. The method of claim 1, wherein the increase in the presence of the M2 macrophages relative to the M1 macrophages causes the wound to heal at an increased rate, increased angiogenesis at the wound, less scarring at the wound, or a combination thereof.

10. The method of claim 1, further comprising:
combining the electrospun structure with a non-electrospun structure.

11. The method of claim 10, wherein the non-electrospun structure is selected from the group consisting of an allograft, a xenograft, a hernia mesh, and a suture.

12. The method of claim 1, wherein the alpha-hydroxy acid is selected from the group consisting of glycolic acid, lactic acid, and a combination thereof.
